(19) United States
(12) Patent Application Publication
(10) Pub. No.: US 2022/0095696 A1
(43) Pub. Date: Mar. 31, 2022

(54) METHOD OF CONTROLLING VAPORIZING DEVICE, VAPORIZING DEVICE, AND CONTROL SYSTEM

(71) Applicant: HUIZHOU HAPPY VAPING TECHNOLOGY LIMITED, Huizhou, Guangdong (CN)

(72) Inventors: Guangrong Lin, Shenzhen, Guangdong (CN); Xianbin Zheng, Shenzhen, Guangdong (CN); Xiyong Zhang, Shenzhen, Guangdong (CN)

(73) Assignee: HUIZHOU HAPPY VAPING TECHNOLOGY LIMITED, Huizhou, Guangdong (CN)

(21) Appl. No.: 17/439,831

(22) PCT Filed: Mar. 23, 2020
(86) PCT No.: PCT/CN2020/080630

§ 371 Date: Sep. 16, 2021

(30) Foreign Application Priority Data
May 21, 2019 (CN) 201910431791.6

(51) Int. Cl. A24F 40/65; A24F 40/53; A24F 40/51; A24F 40/60

(52) U.S. Cl. CPC A24F 40/65; A24F 40/60; A24F 40/51; A24F 40/53

(57) ABSTRACT

The disclosure provides a method of controlling a vaporizing device, a vaporizing device, and a control system. The controlling method comprises communicating with a cloud server, a user terminal device, and a vaporizing device by means of an administrator terminal device, obtaining various parameters of the vaporizing device by means of the administrator terminal device, writing a new control parameter into the vaporizing device by means of the administrator terminal device, and limiting the user's usage by means of the vaporizing device based on the new control parameter. The disclosure has advantages that, the controlling method allows the authorized administrator to login and query the service condition of the vaporizing device by means of the administrator terminal device and to write new control parameters when necessary.

### TECHNICAL FIELD

The disclosure relates to the field of vaporizing devices, more particularly to a method of controlling a vaporizing device, a vaporizing device, and a control system.

### BACKGROUND

The vaporizing device usually heats and vaporizes liquid substances or paste-like substances, such as drugs and E-cigarette liquid, to generate aerosol or vapor for users to use. As people pay more attention to their health, they realize that overuse may be harmful to health.

According to existing methods of controlling a vaporizing device, the users usually can freely use the device without any appropriate restriction or control during using. In particular, the amount of the intake of vaporized aerosol or vapor cannot be calculated, controlled or restricted until the substances to be vaporized, such as drugs and E-cigarette liquid, run out. In such a case, the users may overuse after a long period of use and the excessive intake probably will be harmful to health.

In particular, if users need to use a medicine with the intake or administration dosage controlled by medical personnel, it is necessary for the vaporizing device to be operated under a remote control of a regulator or authorized medical personnel, and it is desired to adjust the limit value of administration dosage in real-time based on user's personal information including physical condition and use condition.

### SUMMARY

### Technical Problem

An object of the disclosure is to overcome the above shortcomings and provide a method of controlling a vaporizing device, a vaporizing device, and a control system. The controlling method can adjust the limit value of administration dosage of the vaporizing device based on user's physical condition and use condition, and the vaporizing device and the control system are designed for implementing the control method.

### Technical Solutions

The disclosure provides a technical solution as follow. A method of controlling a vaporizing device comprises: by a communication between an administrator terminal device and a cloud server, a user terminal device and a vaporizing device, obtaining various parameters of the vaporizing device by means of the administrator terminal device, writing a new control parameter into the vaporizing device by means of the administrator terminal device, and, when the new control parameter is triggered, automatically entering a new state of the vaporizing device to achieve a control for the vaporizing device.

Preferably, the communication between an administrator terminal device and a cloud server, a user terminal device and a vaporizing device may be implemented in such a manner that the administrator terminal device is in communication with the cloud server, the cloud server is in communication with the user terminal device, and the user terminal device is in communication with the vaporizing device.

Preferably, the method may comprise the administrator terminal device and the vaporizing device being in direct communication with each other in a wired or wireless manner.

Preferably, the vaporizing device may be configured with a first wired communication module or a first wireless communication module, wherein the first wired communication module or the first wireless communication module may be in communication with a second wired communication module or a second wireless communication module of the user terminal device, to allow the user terminal device to read information of the vaporizing device and control the vaporizing device.

Preferably, the vaporizing device may be provided with an identification code, by which the user terminal device is capable of creating a connection and communication with the vaporizing device.

Preferably, the cloud server may be configured to store information or a program for identifying the identification code. Once the identification code is input, the administrator terminal device will be authorized to create a communication to the vaporizing device, to obtain various parameters and write the control parameter.

Preferably, the method may further comprise operating steps of:

(1) Opening software of the administrator terminal device, to implement secure logon of an administrator;

(2) Inputting an identification code for the vaporizing device, to enter a parameter setting interface for the vaporizing device;

(3) Reading and analyzing relevant information of the vaporizing device;

(4) Writing a new control parameter based on a service condition of the vaporizing device and user's physical condition;

(5) Storing the new control parameter in the cloud server when the administrator terminal device is in communication with the cloud server;

(6) Storing the new control parameter in the user terminal device when the cloud server is in communication with the user terminal device;

(7) Writing the new control parameter into the vaporizing device when the user terminal device is in communication with the vaporizing device;

(8) Automatically entering a new state of the vaporizing device when the new control parameter is triggered.

Preferably, during the step of automatically entering a new state of the vaporizing device when the new control parameter is triggered, further issuing an alarm by means of the administrator terminal device, or the user terminal device, or the vaporizing device.

Preferably, the new control parameter may comprise a new limit value of aerosol intake, and, when a value of aerosol intake detected by means of the vaporizing device exceeds the new limit value of aerosol intake, shutting down power delivery by means of the power control unit of the vaporizing device for a period of the set OFF time.

Preferably, the limit value of aerosol intake may comprise a limit value of single puff intake. That is, when a value of single puff intake exceeds the limit value of single puff intake, shutting down output by means of the power control unit of the vaporizing device for a period of the set OFF time.

Preferably, the limit value of aerosol intake may comprise a limit value of total intake. That is, when a sum value of aerosol intake per unit time exceeds the limit value of total intake, shutting down output by means of the power control unit of the vaporizing device for a period of the set OFF time.

Preferably, the method may further comprise operating steps of:

(1) Opening software of the administrator terminal device, to implement secure logon of an administrator;

(2) Inputting an identification code for the vaporizing device, to enter a parameter management interface for the vaporizing device;

(3) Reading and analyzing relevant information of the vaporizing device;

(4) Writing a new limit value of single puff intake, a new limit value of total intake and an OFF time based on a service condition of the vaporizing device and user's personal information;

(5) Storing the new limit value of single puff intake, the new limit value of total intake and the OFF time in the cloud server when the administrator terminal device is in communication with the cloud server;

(6) Storing the new limit value of single puff intake, the new limit value of total intake and the OFF time in the user terminal device when the cloud server is in communication with the user terminal device;

(7) Writing the new limit value of single puff intake, the new limit value of total intake and the OFF time into the vaporizing device when the user terminal device is in communication with the vaporizing device;

(8) Implementing controlling of the vaporizing device based on the new limit value of single puff intake, the new limit value of total intake and the OFF time, and, when a value of single puff intake exceeds the limit value of single puff intake or a sum value of aerosol intake per unit time exceeds the limit value of total intake, shutting down output by means of the power control unit of the vaporizing device;

(9) Returning to a standby state of the vaporizing device after a period of the set OFF time passes since output is shut down by means of the power control unit of the vaporizing device.

The disclosure provides another technical solution as follow. A vaporizing device comprises a battery, a control circuit board, a heating resistor, a vaporizing passage, and a mouth piece. The control circuit board is arranged with a microcontroller, a power control unit, and a communication unit, which are configured to execute instructions during operation, to implement the above controlling method. The microcontroller is configured to compare the new control parameter with relevant parameters and then send corresponding control signal to the power control unit. On such basis, the power control unit is configured to control the output power to limit the user's usage.

The disclosure provides another technical solution as follow. A vaporizing device comprises a battery, a control circuit board, a heating resistor, a vaporizing passage, a mouth piece, a detecting air passage, and an air pressure sensor. The air pressure sensor is disposed in the detecting air passage to detect the suction pressure. The control circuit board is further arranged with the microcontroller MCU, an air flow calculating unit, an energy statistics unit, an energy and vaporizing amount conversion unit, and a power control unit, which are configured to execute instructions during operation to implement the above controlling method. The air flow calculating unit is configured to calculate the corresponding value of air flow based on the modeling volume of the detecting air passage and the change of suction pressure. The power control unit is configured to control output power that is output to the heating resistor based on the value of air flow. The energy statistics unit is configured to calculate the energy output to the heating resistor, based on the output power and the period of time. The energy and vaporizing amount conversion unit is configured to calculate the corresponding value of aerosol intake from the energy. The microcontroller is configured to compare the corresponding value of aerosol intake with the preset limit value of aerosol intake and then send corresponding control signal to the power control unit. The power control unit is capable of shutting down for a certain period of time, to limit the aerosol intake.

The disclosure provides another technical solution as follow. A control system for the vaporizing device comprises the vaporizing device, the user terminal device, the cloud server, and the administrator terminal device, which are in communication with each other. The administrator terminal device is configured to obtain various parameters of the vaporizing device during communication, and write the new control parameter into the vaporizing device, and, the vaporizing device is configured to automatically enter a new state to achieve a control for the vaporizing device when the new control parameter is triggered.

Preferably, the user terminal device may be disposed in the vaporizing device.

### Advantages

The method of controlling a vaporizing device according to the disclosure allows the authorized administrator to login and query the service condition of the vaporizing device by means of the administrator terminal device and to write new control parameters when necessary. The method provides the vaporizing device with a targeted remote control in particular on the limit value of aerosol or vapor intake, prevents the user from overusing the aerosol or vapor in single puff or in a unit time, and avoids health hazards. According to the controlling method, the vaporizing device is operated in a condition that it is loaded with control programs. Thus, it is not necessary to remotely control the device on line all the time, and the vaporizing device can be normally operated even without network communication.

### BRIEF DESCRIPTION OF THE DRAWINGS

FIG. 1 is a diagram illustrating communication relation according to a controlling method of the disclosure;

FIG. 2 is a flow chart of a controlling method according to the disclosure;

FIG. 3 is another flow chart of a controlling method according to the disclosure;

FIG. 4 is a cross-sectional view of a vaporizing device according to the disclosure;

FIG. 5 is a functional block diagram of a circuit board of a vaporizing device according to the disclosure;

### DETAILED DESCRIPTION OF ILLUSTRATED EMBODIMENTS

In order to make purposes, technical solutions and advantages of the disclosure clearer, the disclosure will be further explained in detail with reference to drawings and embodiments described hereinafter. It should be understood that the specific embodiments described herein are only used to explain the present invention and are not intended to limit the present invention.

Embodiments

The controlling method of the disclosure provides a remote use and control method which is targeted on the basis that the vaporizing device itself is capable of controlling the user's usage.

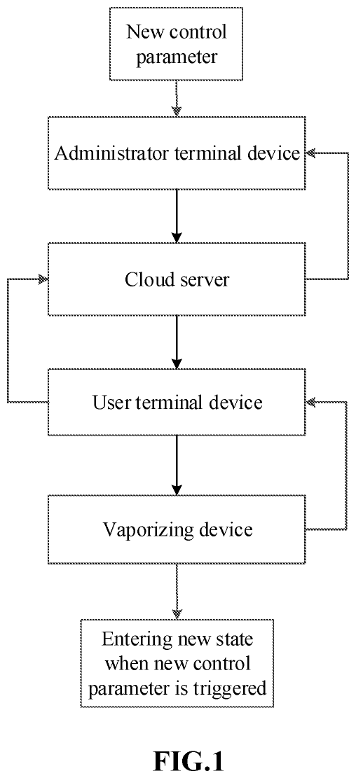

Referring to FIG. 1, a method of controlling a vaporizing device according to the disclosure comprises: by a communication between an administrator terminal device and a cloud server, a user terminal device and a vaporizing device, obtaining various parameters of the vaporizing device and writing a new control parameter into the vaporizing device by means of the administrator terminal device, and, when the new control parameter is triggered, automatically entering a state of the vaporizing device, thereby achieving controlling of the vaporizing device. In an embodiment, the communication between an administrator terminal device and a cloud server, a user terminal device and a vaporizing device is implemented in such a manner that the administrator terminal device is in communication with the cloud server, the cloud server is in communication with the user terminal device, and the user terminal device is in communication with the vaporizing device.

The new control parameter includes a limit value of aerosol intake. In such a case, when a value of aerosol intake detected by means of the vaporizing device exceeds the limit value of aerosol intake, the power control unit of the vaporizing device shuts down power delivery, i.e., the power is cut off and the vaporizing device is stopped. The limit value of aerosol intake includes a limit value of single puff intake and a limit value of total intake. The vaporizing device according to the method of the disclosure are loaded with relevant control programs, and is configured to control, once a new control parameter is written, the user's usage based on the new control parameter.

It is apparent that, the administrator terminal device, the cloud server, the user terminal device, and the vaporizing device according to the method of the disclosure are preinstalled with relevant application and control programs, and have communication function.

Herein, the vaporizing device is configured with a first wired communication module or a first wireless communication module, wherein the first wired communication module or the first wireless communication module may be in communication with a second wired communication module or a second wireless communication module of the user terminal device, to allow the user terminal device to read information of the vaporizing device and control the vaporizing device.

The vaporizing device is provided with an identification code, by which the user terminal device can create a connection with the vaporizing device and create a communication thereto.

The cloud server is configured to store a program for identifying the identification code. Once the identification code is input, the administrator terminal device will be authorized to create a communication to the vaporizing device, such that various parameters may be obtained and the control parameter may be written.

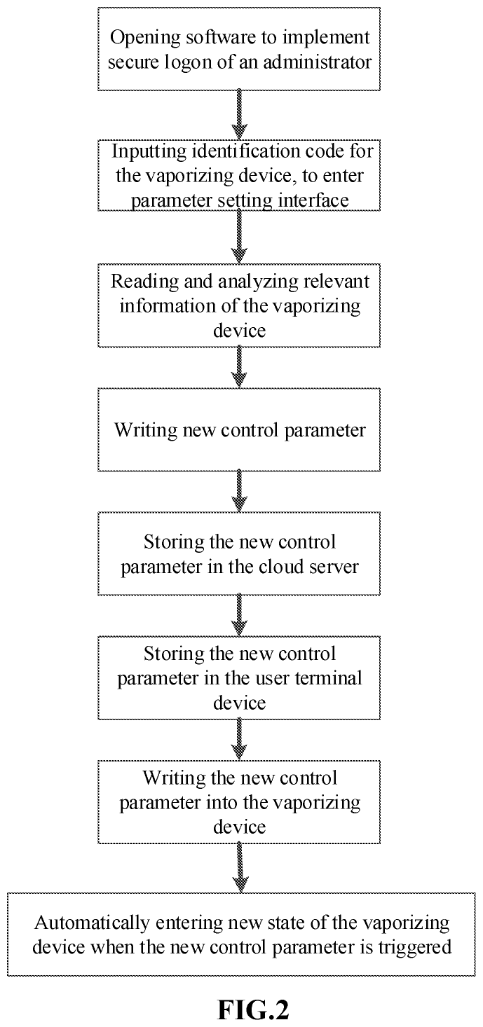

Referring to FIG. 2, a method of controlling a vaporizing device according to the disclosure further comprises steps of:

(1) Opening software of the administrator terminal device, to implement secure logon of administrator;

(2) Inputting an identification code for the vaporizing device, to enter a parameter setting interface for the vaporizing device;

(3) Reading and analyzing relevant information of the vaporizing device;

(4) Writing a new control parameter based on a service condition of the vaporizing device and user's physical condition;

(5) Storing the new control parameter in the cloud server when the administrator terminal device is in communication with the cloud server;

(6) Storing the new control parameter in the user terminal device when the cloud server is in communication with the user terminal device;

(7) Writing the new control parameter into the vaporizing device when the user terminal device is in communication with the vaporizing device;

(8) Automatically entering a new state of the vaporizing device when the new control parameter is triggered.

In another embodiment on the basis of the preceding embodiment, the method further comprises the administrator terminal device and the vaporizing device being in direct communication with each other in a wired or wireless manner. Such method facilitates setting of new parameters on the spot by the administrator having face to face interview with the user.

The method according to the disclosure further comprises controlling the user's usage by means of the vaporizing device according to the new control parameter, and issuing an alarm by means of the administrator terminal device, or the user terminal device, or the vaporizing device when the new control parameter is triggered.

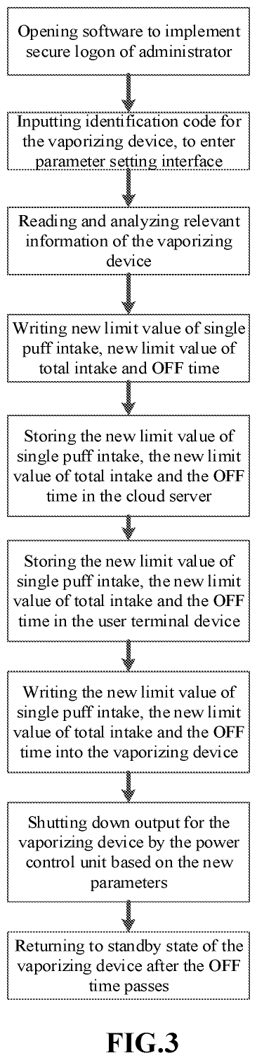

Referring to FIG. 3, in terms of controlling an amount of aerosol intake, or an amount of medicine intake, or an amount of e-cigarette vapor intake, the method according to the disclosure may further comprise steps of:

(1) Opening software of the administrator terminal device, to implement secure logon of administrator;

(2) Inputting an identification code for the vaporizing device, to enter a parameter management interface for the vaporizing device;

(3) Reading and analyzing relevant information of the vaporizing device;

(4) Writing a new limit value of single puff intake, a new limit value of total intake and an OFF time based on a service condition of the vaporizing device and user's personal information;

(5) Storing the new limit value of single puff intake, the new limit value of total intake and the OFF time in the cloud server when the administrator terminal device is in communication with the cloud server;

(6) Storing the new limit value of single puff intake, the new limit value of total intake and the OFF time in the user terminal device when the cloud server is in communication with the user terminal device;

(7) Writing the new limit value of single puff intake, the new limit value of total intake and the OFF time into the vaporizing device when the user terminal device is in communication with the vaporizing device;

(8) Implementing controlling of the vaporizing device based on the new limit value of single puff intake, the new limit value of total intake and the OFF time, and, when a value of single puff intake exceeds the limit value of single puff intake or a sum value of aerosol intake per unit time exceeds the limit value of total intake, shutting down output by means of the power control unit of the vaporizing device;

(9) Returning to a standby state of the vaporizing device after a period of the set OFF time passes since output is shut down by means of the power control unit of the vaporizing device.

In the above steps, the limit value of single puff intake refers to the limit value of the intake such as aerosol, or medicine, or e-cigarette vapor by each single puff of the user. The limit value of total intake refers to a limit value of the sum of aerosol intake per unit time, and the unit time may be set to, for example, the past 24 hours. It is desired to set a limit value for the sum of aerosol intake of the user, to avoid overuse by the user. The OFF time refers to the period of time during which the vaporizing device is automatically shut down in the case that the intake of the user exceeds the limit value of single puff intake or the limit value of total intake, preventing the user from continually using the device in a short time to result in an overuse.

The method of controlling a vaporizing device according to the disclosure allows the authorized administrator (including regulatory authority, authorized medical personnel, and the like) to login and query the service condition of the vaporizing device by means of the administrator terminal device based on the identification code and to write a new control parameter when necessary. The method provides the vaporizing device with a targeted remote control in particular on the limit value of aerosol, medicine, or vapor intake, prevents the user from overusing the aerosol, medicine, or vapor in single puff or in a unit time, and avoids health hazards. According to the controlling method, the vaporizing device is operated in a condition that it is loaded with control programs. Thus, it is not necessary to remotely control the device on line all the time, and the vaporizing device can be normally operated even without network communication.

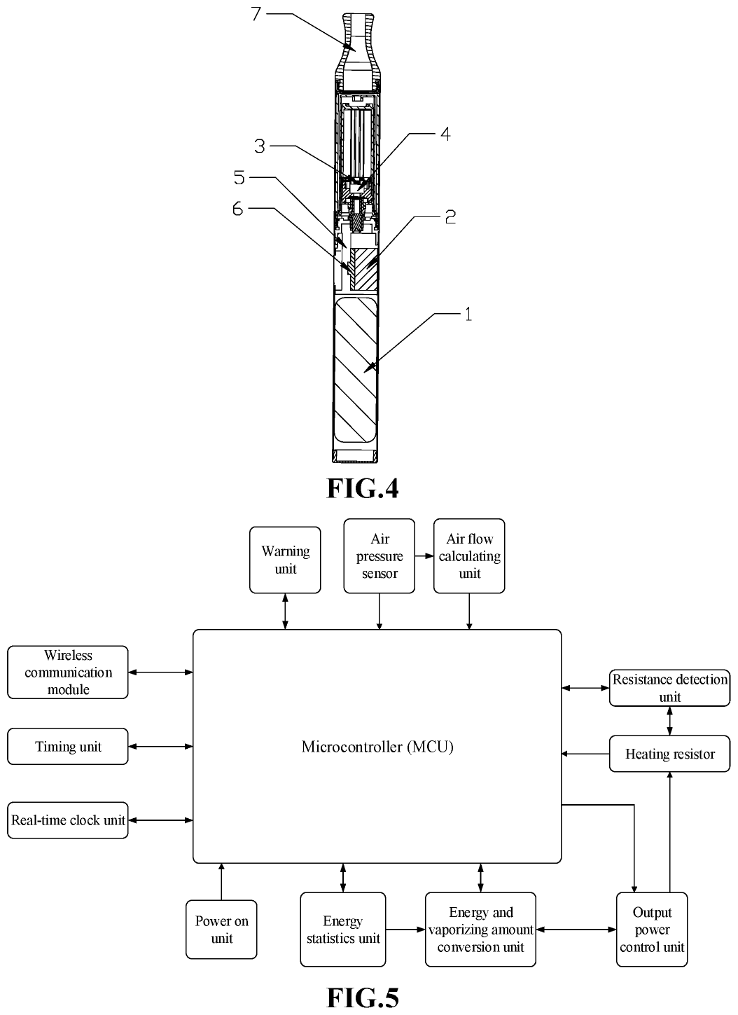

Referring to FIG. 4, the vaporizing device of the disclosure comprises a battery 1, a control circuit board 2, a heating resistor 3, a vaporizing passage 4, and a mouth piece 7. The control circuit board 2 is arranged with a microcontroller MCU, a power control unit, and a communication unit, which are configured to execute instructions during operation, to implement the above controlling method. The microcontroller MCU is configured to compare the new control parameter with relevant parameters and then send corresponding control signal to the power control unit. On such basis, the power control unit is configured to control the output power to limit the user's usage.

Referring to FIG. 4, in the vaporizing device of the disclosure which comprises a battery 1, a control circuit board 2, a heating resistor 3, a vaporizing passage 4, and a mouth piece 7, the heating resistor 3 is disposed in the vaporizing passage 4, and the battery 1 supplies power for the heating resistor 3. Under the control of control circuit board 2, the heating resistor 3 may be energized to produce heat, and then vaporize liquid substances or paste-like substances, to generate aerosol or vapor for inhale. The user may inhale aerosol or vapor through the mouth piece 7. The vaporizing device of the disclosure further comprises a detecting air passage 5 and an air pressure sensor 6 disposed beside the detecting air passage 5 to detect the suction pressure.

Referring to FIG. 5, the control circuit board 2 is arranged with the microcontroller MCU, an air flow calculating unit, an energy statistics unit, an energy and vaporizing amount conversion unit, and a power control unit, which are electrically connected with each other and configured to execute instructions during operation, to implement the above controlling method. The air flow calculating unit is configured to calculate the corresponding value of air flow based on the modeling volume of the detecting air passage and the change of suction pressure. The power control unit is configured to control output power for the heating resistor based on the value of air flow. The energy statistics unit is configured to calculate, based on the output power and the period of time, the energy output to the heating resistor. The energy and vaporizing amount conversion unit is configured to calculate the corresponding value of aerosol intake from the energy. The microcontroller MCU is configured to compare the value of aerosol intake with preset limit value of aerosol intake and then send corresponding control signal to the power control unit. The power control unit is configured to control the output power on such basis, to limit the aerosol intake.

Herein, the corresponding value of aerosol intake may be calculated from the energy by means of the energy and vaporizing amount conversion unit, and then may be compared with the preset limit value of aerosol intake by means of the microcontroller MCU, to determine whether it is close to or exceeds the limit value. Then, the corresponding control signal may be sent to the power control unit. After that, by means of the power control unit, the output power may be reduced or shut down, thereby preventing the user from overusing the medicine, the vapor, or the like. In this way, an automatic control for the aerosol intake of the user is achieved.

The limit value of aerosol intake set in the microcontroller MCU comprises the limit value of single puff intake. When the value of single puff intake exceeds the limit value of single puff intake, the power control unit may shut down the output.

The limit value of aerosol intake set in the microcontroller MCU further comprises the limit value of total intake. When the sum value of aerosol intake per unit time exceeds the limit value of total intake, the power control unit may shut down the output.

Referring to FIG. 1, a control system for the vaporizing device according to the disclosure comprises the vaporizing device, the user terminal device, the cloud server, and the administrator terminal device which are in communication with each other. During communication, the administrator terminal device may obtain various parameters of the vaporizing device, the administrator terminal device may write the new control parameter into the vaporizing device, the vaporizing device may automatically enter a new state when the new control parameter is triggered. In this way, a control for the vaporizing device may be achieved.

According to the disclosure, the administrator terminal device and the user terminal device include but not limited to desktop computers, laptops, smart phones, I-PAD, and the like, which comprise an arithmetic and process unit and a communication unit and can implement the method of the disclosure during operation as long as being installed with specific programs or APPs. Usually, the administrator terminal device may be used by the administrator, i.e., regulatory authority and authorized medical personnel. The user terminal device may be matched with the vaporizing device and used by the user. The vaporizing device may be activated only when the user logs in the user terminal device and the identification code is matched, thereby avoiding misuse by others or minors.

The vaporizing device of the disclosure may limit the usage of the user according to the new control parameter. When the new control parameter is triggered, the alarm may be issued by means of the administrator terminal device, or the user terminal device, or the vaporizing device. That is, these devices may comprise an alarm unit. In addition, in the event of power on and off, stand-by time, the single puff intake exceeding the limit value, the sum intake exceeding the limit value, overheat, overload, or dry-heating, and the like, the alarm unit may warn the user by an alarm in the form of a sound, a light, a vibration, and the like.

In another embodiment, the user terminal device may be simplified and disposed in the vaporizing device, i.e., the user terminal device and the vaporizing device are incorporated, such that the vaporizing device has the function of the user terminal device, thereby facilitating usage and operation of the user.

### INDUSTRIAL APPLICABILITY

All the above are merely preferred embodiments of the disclosure. The present invention is intended to cover all modifications and equivalent arrangements those skilled in the art can make according to the technical essence of the present invention.

1. A method of controlling a vaporizing device, wherein the method comprises: by a communication between an administrator terminal device and a cloud server, a user terminal device and the vaporizing device, obtaining various parameters of the vaporizing device by means of the administrator terminal device, writing a new control parameter into the vaporizing device by means of the administrator terminal device, and, automatically entering a new state of the vaporizing device to achieve a control for the vaporizing device when the new control parameter is triggered.

2. The method of controlling a vaporizing device according to claim 1, wherein the communication between an administrator terminal device and a cloud server, a user terminal device and a vaporizing device is implemented in such a manner that the administrator terminal device is in communication with the cloud server, the cloud server is in communication with the user terminal device, and the user terminal device is in communication with the vaporizing device.

3. The method of controlling a vaporizing device according to claim 1, wherein the method comprises the administrator terminal device and the vaporizing device being in direct communication with each other in a wired or wireless manner.

4. The method of controlling a vaporizing device according to claim 1, wherein the vaporizing device is configured with a first wired communication module or a first wireless communication module, wherein the first wired communication module or the first wireless communication module is in communication with a second wired communication module or a second wireless communication module of the user terminal device, to allow the user terminal device to read information of the vaporizing device and control the vaporizing device.

5. The method of controlling a vaporizing device according to claim 1, wherein the vaporizing device is provided with an identification code, by which the user terminal device is capable of creating a connection and communication with the vaporizing device.

6. The method of controlling a vaporizing device according to claim 5, wherein the cloud server is configured to store information or a program for identifying the identification code, the administrator terminal device is configured to be authorized by inputting the identification code, to create a communication with the vaporizing device, to obtain various parameters and write the control parameter.

7. The method of controlling a vaporizing device according to claim 1, wherein the method further comprises operating steps of:
(1) opening software of the administrator terminal device, to implement secure logon of an administrator;
(2) inputting an identification code for the vaporizing device, to enter a parameter setting interface for the vaporizing device;
(3) reading and analyzing relevant information of the vaporizing device;
(4) writing a new control parameter based on a use condition of the vaporizing device and user's personal information;
(5) storing the new control parameter in the cloud server when the administrator terminal device is in communication with the cloud server;
(6) storing the new control parameter in the user terminal device when the cloud server is in communication with the user terminal device;
(7) writing the new control parameter into the vaporizing device when the user terminal device is in communication with the vaporizing device;
(8) automatically entering a new state of the vaporizing device when the new control parameter is triggered.

8. The method of controlling a vaporizing device according to claim 1, wherein during the step of automatically entering a new state of the vaporizing device when the new control parameter is triggered, further issuing an alarm by means of the administrator terminal device, or the user terminal device, or the vaporizing device.

9. The method of controlling a vaporizing device according to claim 1, wherein the new control parameter comprises a new limit value of aerosol intake, and, when a value of aerosol intake detected by means of the vaporizing device exceeds the new limit value of aerosol intake, shutting down power output by means of the power control unit of the vaporizing device for a period of set OFF time.

10. The method of controlling a vaporizing device according to claim 9, wherein the limit value of aerosol intake comprises a limit value of single puff intake, that is, when a value of single puff intake exceeds the limit value of single puff intake, shutting down output by means of the power control unit of the vaporizing device for the period of set OFF time.

11. The method of controlling a vaporizing device according to claim 9, wherein the limit value of aerosol intake comprises a limit value of total intake, that is, when a sum value of aerosol intake per unit time exceeds the limit value of total intake, shutting down output by means of the power control unit of the vaporizing device for the period of set OFF time.

12. The method of controlling a vaporizing device according to claim 1, wherein the method further comprises operating steps of:
(1) opening software of the administrator terminal device, to implement secure logon of an administrator;
(2) inputting an identification code for the vaporizing device, to enter a parameter management interface for the vaporizing device;
(3) reading and analyzing relevant information of the vaporizing device;
(4) writing a new limit value of single puff intake, a new limit value of total intake and an OFF time based on a service condition of the vaporizing device and user's personal information;
(5) storing the new limit value of single puff intake, the new limit value of total intake and the OFF time in the cloud server when the administrator terminal device is in communication with the cloud server;
(6) storing the new limit value of single puff intake, the new limit value of total intake and the OFF time in the user terminal device when the cloud server is in communication with the user terminal device;
(7) writing the new limit value of single puff intake, the new limit value of total intake and the OFF time into the vaporizing device when the user terminal device is in communication with the vaporizing device;
(8) implementing controlling of the vaporizing device based on the new limit value of single puff intake, the new limit value of total intake and the OFF time, and, when a value of single puff intake exceeds the limit value of single puff intake or a sum value of aerosol intake per unit time exceeds the limit value of total intake, shutting down output by means of the power control unit of the vaporizing device;
(9) returning to a standby state of the vaporizing device after the period of set OFF time passes since output is shut down by means of the power control unit of the vaporizing device.

13. A vaporizing device, comprising a battery, a control circuit board, a heating resistor, a vaporizing passage, and a mouth piece, wherein the control circuit board is arranged with a microcontroller, a power control unit, and a communication unit, which are configured to execute instructions during operation to implement the controlling method according to claim 1, the microcontroller is configured to compare relevant parameters with a new control parameter and then send corresponding control signal to the power control unit, and the power control unit is configured to control, on such basis, output power to limit user's usage.

14. A vaporizing device, comprising a battery, a control circuit board, a heating resistor, a vaporizing passage, a mouth piece, a detecting air passage, and an air pressure sensor disposed in the detecting air passage to detect suction pressure, wherein the control circuit board is further arranged with a microcontroller, an air flow calculating unit, an energy statistics unit, an energy and vaporizing amount conversion unit, and a power control unit, which are configured to execute instructions during operation to implement the controlling method according to claim 9, the air flow calculating unit is configured to calculate corresponding value of air flow based on a modeling volume of the detecting air passage and a change of suction pressure, the power control unit is configured to control output power that is output to the heating resistor based on the value of air flow, the energy statistics unit is configured to calculate energy output to the heating resistor based on the output power and the period of time, the energy and vaporizing amount conversion unit is configured to calculate the corresponding value of aerosol intake from the energy, the microcontroller is configured to compare the corresponding value of aerosol intake with a preset limit value of aerosol intake and then send corresponding control signal to the power control unit, the power control unit is capable of shutting down power output for a certain period of time to limit aerosol intake.

15. A control system for a vaporizing device, wherein the control system comprises the vaporizing device, a user terminal device, a cloud server, and an administrator terminal device, which are in communication with each other, the administrator terminal device is configured to obtain various parameters of the vaporizing device during communication and write a new control parameter into the vaporizing device, and the vaporizing device is configured to automatically enter a new state to achieve a control for the vaporizing device when the new control parameter is triggered.

16. The control system for a vaporizing device according to claim 15, wherein the user terminal device is disposed in the vaporizing device.
